(19) United States
(12) Patent Application Publication
(10) Pub. No.: US 2011/0244028 A1
(43) Pub. Date: Oct. 6, 2011

(54) METHOD OF SOLUBILIZING BIOLOGICALLY ACTIVE COMPOUNDS

(76) Inventors: Steve Leigh, Muttenz (CH); Mathew Leuis Steven Leigh, Muttenz (CH); Peter Van Hoogevest, Bubendorf (CH)

(21) Appl. No.: 13/121,042

(22) PCT Filed: Sep. 28, 2009
(86) PCT No.: PCT/EP2009/062543

§ 371 Date: Jun. 8, 2011

(30) Foreign Application Priority Data
Sep. 26, 2008 (EP) 08165306.5

(51) Int. Cl. A61K 9/127; A61K 31/5513; A61K 31/519; A61K 31/496; A61K 31/4164; A61P 43/00

(52) U.S. Cl. 424/450; 514/221; 514/262.1; 514/254.07; 514/385

(57) ABSTRACT

The invention describes a method of preparing lipid compositions by associating pH sensitive compounds which maybe weak acids, bases or amphoteric compounds with at least one ionisable or pH sensitive group and low water solubility into preformed and standardised lipid particles by pH manipulation. Drug association is carried out in situ by changing the pH of the aqueous medium to pH 6-8 wherein the compound spontaneously partitions into the lipid particles. Drug association may also be carried out by exploiting pH changes in the GI tract. The lipophilic compound is solubilised by the lipid particles and may be used as such for in-vitro and pharmacokinetic assessments or processed for oral, parenteral or topical therapy.

### CROSS-REFERENCE TO RELATED APPLICATIONS

This application claims priority to PCT Patent Application No. PCT/EP2009/062543 filed on Sep. 28, 2009 and European Patent Application No. 08165306.5 filed on Sep. 26, 2008, the entirety of each of which is incorporated by this reference.

### BACKGROUND OF THE INVENTION

1. Field of the Invention

This invention relates to a method of solubilizing biologically active compounds.

2. State of the Art

A problem in administering and testing biologically active compounds in living organisms concerns low water solubility at physiological pH, particularly blood pH and GI tract pH. Solubility influences drug concentrations in biological fluids which reach the target organ. It is essential that a compound is solvated to facilitate diffusion and transport across biological membranes. Generally, poorly soluble compounds that have at least one acidic and/or basic function are more water soluble as salts or when ionized. Unionized forms are more soluble in lipid membranes and organic solvents and more able to cross lipid cell membranes for drug absorption. A class of compounds which is particularly difficult to administer intravenously (parenterally) are compounds wherein the ionized or salt form is more soluble in water at non-physiological pH values (i.e. either >pH 8 or <pH 6) and the unionized or free form is less soluble at physiological or blood pH (ca. pH 7.2-7.4). Injectables with a pH of either pH 8 or <pH 6 wherein the compound is partially or completely dissolved causes pain at the injection site. Furthermore, dilution with blood at pH 7.2-7.4 may precipitate the drug in the solution, reducing pharmacological activity and risking phlebitis and emboli. Drugs which are insoluble at either >pH 8 or <pH 6, or at pH 6 to pH 8 may not be suitable for intravenous injection as such and alternative dosage forms or non-parenteral therapy may have to be considered. After oral administration, basic compounds may be solubilized in the stomach where the pH is ca. 2 and precipitate on reaching lower parts of the GI tract where the pH is higher.

Lipid dispersions such as liposomes, micro emulsion droplets, micelles and mixed micelles are employed to solubilize or associate with lipophilic compounds. Liposomes are selectively used in drug therapy for carrying poorly soluble compounds in a solubilized form and to study the effects of the compound in living organisms, for example in toxicity, ADME pharmacokinetic and clinical studies during drug development. Compounds transported in lipid particles may give better bioavailability because of higher blood concentrations and improved uptake by targeted organs.

A further problem concerns formulation development and preclinical trials where solvents and/or intensive mechanical energy input are required for solution or association in lipid dispersions such as liposomes, oil-in-water emulsions and surfactant based systems. Micro fluidizers and high pressure homogenizers do not generate sufficient energy for molecular solution in the absence of a suitable solvent and medium. Solvents may not be tolerated even in small amounts notwithstanding the possibility of solvent removal. Expediency, safety and reproducibility are major considerations in studies and in vivo screening tests which extend to first use and exposure in humans. There is as yet an unmet need in early stage development to identify the most suitable carrier to solubilize or associate with poorly soluble compounds which facilitates preliminary in vivo trials in the absence of a comprehensively developed and tested formulation.

ER-B-O 158 441 relates to pro-liposome compositions based on membrane lipids, to a method of making lipid vesicles by the addition of aqueous medium to the compositions and to aqueous dispersions of vesicles. The compositions may contain water or oil soluble biologically active compounds which are encapsulated in the aqueous phase of the liposomes or associated with the lipids during spontaneous formation of liposomes. It does not involve using acid or alkaline pH conditions to associate unionized compounds with the lipid in an aqueous medium without involving a volatile organic solvent.

WO97/25977 discloses a process for preparing an oil-in-water fat emulsion composition containing a cyclosporin, a rapamycin or an ascormnycin or a derivative thereof, by mixing a placebo fat emulsion with a concentrate comprising the active compound, a stabilizer (e.g. phospholipid) and an organic solvent.

U.S. Pat. No. 5,192,549 discloses a method of amphipathic drug loading into liposomes by applying a permanent pH gradient, whilst U.S. Pat. No. 5,316,771 describes amphipathic drug loading into the aqueous compartment of the liposomes by ammonium ion gradient. U.S. Pat. No. 5,380,531 also describes a method for an accumulation of amino acids and peptides into liposomes. The examples are confined to the loading of the aqueous compartment of the liposomes with hydrophilic very water soluble compounds at pH 7.2 (i.e. the compound is not in dispersion) possessing a basic function which can be ionized.

U.S. Pat. No. 5,616,341, describes high drug lipid ratios in formulations comprising liposomal antineoplastic agents. The liposomes may be made by a process that loads the drug by an active mechanism using a trans membrane ion gradient, such as a trans c Membrane pH gradient between the inside and outside of the lipid bilayers. Trapping efficiencies in the aqueous compartments of the liposomes are claimed to approach 100%. However, the method disclosed is restricted to loading water soluble (at pH 7.2) and ionizable antineoplastic agents in the internal aqueous domain of the vesicles. The drug permeates the membrane and because of the pH gradient created, the more soluble salt form enriches the aqueous compartments, rather than associate with the lipid layers of the liposomes,

EP-B-O 560 138 describes the association of lipophilic drugs with liposomes by co- mixing the lipids and lipophilic drugs and other excipients, e.g. pH stabilizers, relying on high shear homogenization without involving pH manipulation. Aside from the use of higher temperatures (75° C.), which may be deleterious to the compound, it is silent on the use of acid or alkaline pH to associate the lipophilic compound with the lipid phase.

EP 0 974 364 A1 discloses a method to inhibit precipitation of drug solutions for infusion by adding lipid compositions to suppress precipitation. The method does not teach pH adjustment for solubilizing poorly soluble compounds into preformed lipid particles.

U.S. Pat. No. 6,838,090 describes dosage forms wherein a compound with low water solubility is dissolved in organic solvents and added to liposome dispersions in a separate container.

WO 02/080883 describes a method of associating poorly soluble compounds in small unilamellar vesicles (SUV) dispersions characterized by optical clarity. The method describes a two container system wherein a lyophilized powder or a solution in hydrophilic solvents in a first container is mixed with the contents in a second container containing a dear dispersion of SUVs. The application is silent on the use of pH to associate the poorly soluble compounds with SUVs,

WO 9632930 discloses a process which loads pH sensitive weak acid compounds into the aqueous compartments of liposomes. Proton shuttle and the resulting pH gradient formed between the internal and external aqueous medium facilitate higher concentration of the weak-acid salt form inside liposomes. The method lacks the sequence of steps whereby the poorly-soluble free acid form is liberated in the external aqueous phase of the liposome suspension allowing the unionized drug to transfer to lipid domains, not pH driven into internal aqueous phase.

WO 9833484 discloses a method of preparing liposome having a pH sensitive ionizable hydrophobic compound, by forming liposomes in a first buffered aqueous medium containing a base, adding an ionizable (acidic) hydrophobic compound such as Taxol and an acid, thereby creating a pH gradient and inducing the anionic hydrophobic compound to accumulate as the salt form in the liposomes' internal aqueous phase. However, the poorly soluble compound is added after the pH of the external liposome phase has been lowered and the compound is not essentially located in the lipid domain.

U.S. Pat. No. 5,814,335 describes liposomes for delivery of an alkaloid such as vincristine in a first buffered aqueous solution in acidic pH and suspending the liposomes in a second buffered solution at a greater pH than the first to transfer the salt form to the interior aqueous spaces. The sequence of steps involved does not result in dissociation of the salt form between pH 6 and 8 in the external aqueous medium and transfer the liberated lipophilic form to lipid particles.

U.S. Pat. No. 6,083,530 relates to liposomes containing either an acid or a base for internalizing the salt form of basic or acid compounds. The external medium for a basic compound is raised to create a higher gradient of (H +) ions inside the internal aqueous phase, either by adding a base or by dialysis. As a result the basic compound is transferred across the concentration gradient and held as the salt form in the internal aqueous phase.

U.S. Pat. No. 5,082,664 concerns liposomes containing a prostaglandin loaded by a transmembrane concentration gradient method. Lipid and prostaglandin are co-dissolved in an aqueous-miscible organic solvent and then added slowly to a first aqueous solution such as citrate or phosphate. The disclosure involves obtaining a liposome suspension comprising water-miscible solvents, drying protectants and the prostaglandin which is water-soluble at pH 7. The prostaglandin is trapped in the aqueous spaces formed by the liposome membranes.

The prior art disclosures do not describe a method of solubilizing pH sensitive poorly water soluble compounds in lipid-soluble forms at a pH between 6 and 8. The known methods generally employ pH gradients to generate a driving force between external aqueous medium and internal aqueous spaces thereby accumulating the more water- soluble salt form inside the aqueous compartment of liposomes. The disclosures teach away from the subject matter of the invention whereby the drug is believed to internalize within lipid domains rather than internal aqueous spaces. Further, the prior art teaches containment in liposomes by applying a sequence of steps which transfer the drug by pH gradients between external and internal aqueous medium. Whereupon it is claimed, the drug is gradually released from solution. In sharp contrast, the present invention describes a sequence of inventive steps whereby the lipophilic compound appears to dissociate at a pH between 6 and 8 in the external medium whereupon the unionized fraction spontaneously partitions essentially in the lipophilic domains rather than within aqueous spaces. Thus the known methods essentially lack the sequence of steps which dissociate ionizable compounds between pH 6 and 8 in the external medium after the drug is added and the pH changed to acid or alkali to obtain the salt form, or the drug is added in acid or alkali solution. A further contrast concerns the use of preformed or standardized blank or empty lipid vesicles that are ready prepared in bulk and may be conveniently used as such from stock. The lipid dispersions conform to physical standards such as particle size and lamellarity which facilitate reproducible entrapment of a wide array of lipophilic compounds. By comparison the prior art liposome suspension for pH gradients or trans membrane potential transfer require that either the liposomes are prepared with the compound (U.S. Pat. No. 5,082,664), or an acid or a base (WO 9632930 and WO 9833484) is trapped within the liposomes before adding the compound.

In the specification, the following definitions shall apply:

“ Lipid” as used herein refers to various types of polar lipid namely, phospholipids, glycolipids, ceramides, gangliosides and cerebrosides, sphingomyelins, triglycerides, diglycerides, monoglycerides, bile acids and salts thereof, e.g. sodium taurocholate, sodium glycocholate, “charged lipid” cardiolipins, fatty acids, phosphatidylglycerols, phosphatidylinositols, glycolipids, phosphatidic acid, phosphatidylserines, and cationic lipids.

“Lipid dispersion” refers to biphasic systems consisting of lipid particles which may be vesicular (e.g. liposomes or cubosomes) or non vesicular; or mixtures of vesicular and non vesicular lipid particles. The non vesicular lipid particles may be micelles, mixed micelles, oil-in-water type micro or macro emulsions; or mixed compositions thereof.

“Compound ” or “drug” are biologically active, poorly water soluble compounds with one or more ionizable or pH sensitive groups that have physiological and/or pharmacological effects in living organisms and may be utilized for therapeutic administration; or subjected to in vitro or in vivo absorption (e.g. for absolute bioavailability testing)_{;} distribution, metabolism, elimination (ADME) studies including toxicity evaluations,

“Low water solubility” or “poorly soluble” defines a compound that is lipophilic, hydrophobic or amphipathic and requires more than 100 parts by weight of water to dissolve 1 part by weight of the compound at a pH between pH 6 and pH 8. The term particularly applies to very slightly soluble compounds (requiring from 1'000 to 10'000 parts by weight of water to dissolve 1 part by weight of the compound) and practically insoluble or insoluble compounds requiring more than 10'000 parts by weight of water to dissolve 1 part by weight of the compound at a pH between pH 6 and pH 8.

“pH sensitive compound” is ionizable and has low water solubility at “neutral” pH between pH 6 and pH 8 and increased solubility in acid and/or alkaline conditions.

“ Loading” or “transfer” means complexing or associating the poorly soluble pH sensitive compound in the aqueous medium with a lipid dispersion wherein at least 50% by weight of said compound is associated, partitioned or dissolved in the lipid domains of the particles. Maximum lipid association is obtained wherein the optical transmission or clarity of the loaded lipid dispersion at equilibrium is at least 30% of the value of the blank (unloaded) lipid dispersion prior to addition of the suspension or solution of the drug.

“Lipid associates” are complexes formed by molecular or colloidal association between the pH sensitive lipophilic form and lipid particles dispersed in the aqueous medium.

Association or solubilization of the compound in lipid at equilibrium, under the conditions employed is satisfactorily achieved if at least 30% by weight of the compound remains solubilized in the lipid. The unassociated particles of the compound are retained on a 200 nm pore size polycarbonate membrane filter after filtration of the loaded lipid dispersion.

As used herein, the singular term includes the plural and the plural term imports the singular.

Advantages of the present invention are to provide a reproducible and up-scalable method for associating poorly soluble compounds in (the lipid domain of) lipid particles dispersed in aqueous medium and to provide formulations for pH sensitive poorly water soluble compounds which are isohydric and prevent or decrease the risk for drug precipitation at the injection site. The aqueous dispersion containing lipid particles and associated compound at a pH between pH 6 and pH 8 is particularly suitable as delivery or dosing vehicle for intravenous and parenteral injection. It is also used as such for pulmonary and occular delivery of poorly water soluble compounds.

Alternatively, the principle may be applied for delivery of pH sensitive poorly water soluble compounds in oral dosage forms, by exploiting the natural pH changes in the GI tract as the contents digest in transit from acid pH (<4) in the stomach to the higher pH (>4) regions of the intestine, reaching between pH 6 and pH 8, commonly recognized as the absorption window for many drugs.

### SUMMARY OF THE INVENTION

The invention is in the area “poorly soluble compounds”, “pH manipulation”, and “spontaneous lipid solubilization”.

This disclosure presents an inventive method which spontaneously associates poorly water soluble compounds in preformed lipid particles dispersed in the aqueous medium by manipulating the pH of the aqueous medium. The compositions obtainable by the inventive method are suitable as solubilizing vehicle or carrier for in vitro studies, in vivo toxicity tests, ADME tests and for clinical applications. The method avoids or reduces the amount of solvents other than water and favors pH manipulation of the aqueous medium to spontaneously associate at least 30%, or at least 80% by weight of the compound in optically clear lipid dispersions. A feature of the invention is that water miscible organic solvents such as ethanol, DMSO and NMP are not essential to solubilize the compound. Therefore, the removal of residual solvents does not present an issue for applications requiring solvent-free drugs.

Suitable compounds are lipophilic bases, acids or amphoteric and contain at least one ionizable or pH sensitive group which can form acid or base salts that are more water soluble under acidic conditions (pH &It; 6) or basic conditions (pH &gt; 8) within the definition of low water solubility above. Furthermore the degree of dissociation in water is sufficiently high for at least about 30% by weight of the unionized drug to associate with the lipid when the pH of the external aqueous medium is adjusted to pH 6-8.

Thus the pH for forming the more soluble salt form is pH &It; 6 or pH &gt; 8. For spontaneous lipid association, the pH of the aqueous medium of the lipid dispersion is adjusted to between pH 6 and pH 8.

The inventive method exploits the principle that salvation is essential for ionization or dissociation. At pH values below about 6 for basic compounds and above about 8 for acid, the salt form is more soluble. The unionized and ionized forms of the compound are in equilibrium in the solution and the concentrations depend on the pH of the aqueous medium and the pKa of the compound. When the pH of the solution is changed or restored to between pH 6 and pH 8 the degree of ionization/dissociation is affected. The principles of the present invention are employed to manipulate the dissociation in the external aqueous medium by adjusting the pH of the compound in suspended lipid particles in situ, thereby spontaneously transferring the liberated (neutral) lipophilic fraction into lipid compartments, where the compound is associated (solubilized) presumably because of higher lipid water partition coefficient (log P). The method is surprisingly facile and to the best of knowledge has not been previously described to solubilize poorly water-soluble and pH sensitive lipophilic compounds, i.e. by external pH manipulation without creating pH gradients between internal and external aqueous medium.

By the method of the invention, injectable liquid compositions may be prepared for intravenous (parenteral) use as well as for oral therapies and topical therapies which include pulmonary and occular applications. The aqueous dispersion containing drug associated lipid particles may be used as such for therapeutic applications and/or to carry out pharmacokinetic assessments. Alternatively, by removing water by lyophilization or freeze drying, solid carrier compositions with improved long term storage properties are prepared in sealed unit containers. The freeze dried or lyophilized compositions containing the drug may be re-constituted by adding aqueous medium from a second container. The compositions may be prepared aseptically or end sterilized.

pH manipulation may also occur spontaneously wherein the solid carrier comprising a pH sensitive compound dispersed or partly dispersed with phospholipids in dry or hydrated form transits after oral administration, starting from the stomach where the pH of the aqueous environment maybe as low as pH 2 up to between about pH 6 and pH 8 in the duodenum and lower GI tract regions.

Variants of the method and alternative embodiments for preparing lipid dispersions comprising poorly soluble compounds particularly suited for intravenous and parenteral applications are: According to the invention the method of solubilizing a biologically active poorly water soluble compound with one or more ionizable or pH sensitive groups comprises the steps of

a) preparing a lipid dispersion in a first aqueous medium;

b1) dispersing or suspending the poorly water soluble compound in said first aqueous medium;

c1) increasing or lowering the pH of the aqueous medium so that a higher amount of the compound is being dissolved than at essentially neutral conditions;

or alternatively;

b2) dispersing or suspending the poorly water soluble compound in a second aqueous medium;

c2) increasing or lowering the pH of said second medium so that a higher amount of he compound is dissolved than at essentially neutral conditions; and

d) mixing said first medium or said first and second media; and

e) lowering or increasing the pH of the medium again to transfer the compound to the lipid particles and form a lipid dispersion loaded with the compound, in particular a lipid dispersion in which the compound is mainly associated with the lipid domain of the lipid particles.

In one embodiment the poorly soluble compound may be a powder suspended in aqueous medium at around pH 7 containing preformed lipid particles before changing the pH to below pH 6 for basic compounds or above pH 8 for acid compounds to obtain the more soluble salt form in situ in the aqueous medium.

In one embodiment the preformed blank lipid dispersion comprising lipid particles may be at the desired acid pH below 6 for basic compounds or above pH 8 for acid compounds to obtain the more soluble salt form in aqueous solution after adding the poorly soluble powder.

In a further variant the more soluble salt form may be prepared separately or independently in acid pH medium below pH 6 for basic compounds or pH medium above pH 8 for add compounds as a solution or as a powder processed from the solution before adding or combining with the preformed lipid dispersion.

In one embodiment the previously prepared lipid dispersion may contain a buffer with sufficient buffering capacity so that after adding the poorly water soluble compound dissolved or partially dissolved in acid or alkaline solution (below pH 6 or above pH 8) or in a salt form, the buffer adjusts the pH to between pH 6 and pH 8 for spontaneous lipid association in (with) the lipid particles.

Alternatively, if the pH of the solution which is required to dissolve the basic compound is approximately more than at least two pH units below pH 6, possibly more than 4 pH units, or for acid compound the pH is at least 2, possibly above 4 pH units above pH 8, it may be sufficient if the pH of the resulting lipid dispersion after adding the compound is raised or lowered by the buffer, by about one or two pH units to associate said compound with the lipid particles.

According to another aspect of the invention there may be provided a kit for solubilizing a biologically active compound comprising a first container with the lipid dispersion and a second container with the compound dissolved in alkaline or acid medium. Such a kit can be employed in but not limited to diagnostic, therapeutic, or other applications covering e.g. CNS, oncology, cardiovascular diseases, metabolic, dermatology and antibiotic therapy, infectious diseases, etc. Advantageously, a third vial or container with acid, alkaline or buffered medium is used. The contents of the containers can be combined for solubilizing the pH sensitive compound by transferring it to the dispersed lipid particles.

Further embodiments suitable for solid dosage forms include: An embodiment wherein at least one pH change is carried out in a separate step according to the method described, followed by the subsequent removal of the first aqueous medium, and a final pH change is carried out in a further step between pH 6 and pH 8 in intestinal fluid or biorelevant media thereby solubilizing at least 30% by weight of said poorly water soluble compound in lipid particles in said fluid medium.

In one embodiment the pH sensitive compound is dispersed or partially dispersed in a solid carrier together with at least one lipid or lipid particles by removing the water after the first pH change thereby defining a solid composition, further comprising a step of dissolving said composition in an aqueous medium which may be gastric or simulated gastric medium, followed by further pH changes wherein the final pH change is carried out between pH 6 and pH 8 in intestinal fluid or biorelevant media thereby solubilizing at least 30% by weight of said poorly water soluble compound in lipid particles in said fluid medium.

In an alternative embodiment the aqueous medium is removed after the first pH change so that a solid composition comprising lipid or lipid particles, optionally including carrier substances, is obtained, further comprising a step of dissolving said composition in an aqueous medium which may be in simulated gastric fluid or in vivo gastric fluid, followed by further pH changes wherein the final pH change is carried out between pH 6 and pH 8 in intestinal fluid in vivo or biorelevant media thereby solubilizing at least 30% by weight of said poorly water soluble compound in lipid particles in said fluid medium.

In yet another embodiment a pH sensitive compound which is poorly water soluble at a pH between pH 6 and 8 dispersed or partially dispersed in a liquid carrier together with at least one lipid or lipid particles wherein at least one pH change is carried out in a first liquid medium which may be gastric or simulated gastric medium and the final pH change is carried out between pH 6 and pH 8 in a second liquid medium consisting of intestinal fluid or biorelevant media thereby solubilizing at least 30% by weight of said poorly water soluble compound in lipid particles in said fluid medium.

### DETAILED DESCRIPTION OF THE INVENTION

The invention describes a method of solubilizing, pH sensitive compounds with low water solubility by pH manipulation in optically dear lipid particles. The compounds maybe weak acids, bases or amphoteric compounds with at least one ionisable or pH sensitive group. Solubilization is carried out in situ in the lipid by changing or adjusting the pH of the aqueous medium to spontaneously partition the lipophilic form in the lipid particles. The dispersion containing the compound in a solubilized form may be sterilized by aseptic filtration or end sterilization depending on the stability of the compound. The resulting composition is suitable as such for parenteral use, including intravenous and parenteral injection, pulmonary and occular delivery and in vitro and in vivo pharmacokinetic assessments. Alternatively, water may be removed from the aqueous medium by e.g. lyophilization, spray drying or evaporation and the like.

Pharmaceutical excipients such as sugars may be added prior to removal of the water to maintain the original size of the lipid particles containing the solubilized form of the compound.

Poorly water soluble compounds may be crystalline or amorphous powders or mixtures and compositions thereof. The particles are below 100 μm, below 5 μm mean diameter, or between 0.1 μm to 3 μm.

The powder particles may be added to preformed lipid dispersions, optically dear and in a suitable container or individual unit containers such as single or multi-dose glass vials with silicone rubber closures and the like.

Alternatively the compound is dissolved or partially dissolved in acid solution below about pH 6 or alkaline solution above about pH 8 in a separate container and added to the lipid dispersion.

Amphoteric compounds may be treated either as an acidic or basic compound depending on higher solubility in acid or alkaline pH.

The lipid dispersion is previously prepared. The liposomes or alternative forms of lipid particles are characterized in terms of optical clarity, type of lipid, particle size, and lamellarity. The dispersion may be neutral (around pH 7) or at a desired pH, below pH 6 or above pH 8, by including acid, alkali or buffers. Alternatively, the lipid dispersion may be formed in-situ from a substantially anhydrous hydrophilic lipid matrix comprising e.g. phospholipids or liposome precursors.

The pH of the lipid dispersion may be adjusted to the desired pH between pH 6 and pH 8 before or after adding the compound. It should be appreciated that the invention describes a series of inventive steps whereby—without being bound by the explanation it is likely that the lipophilic compound dissociates in the external phase in the presence of lipid particles at a pH between 6 and 8 after the drug is added, culminating in spontaneous transfer to lipophilic domains and not internal aqueous spaces. The principle described herein targets forming the more soluble salt solution of the drug below pH 6 for basic compounds and above pH 8 for acid compounds. By adjusting the liposome dispersion in situ to between pH 6 and 8 the dissociated lipophilic form of the drug partitions in the lipid compartments. As such the order in the manipulation steps of the pH in the lipid dispersion may be varied as long as the method results in formation of the more soluble solution of the compound in acid or alkaline pH. Subsequent adjustment of the dispersion—to about pH 6 to pH 8 transfers the dissociated compound to lipid particles in situ. Prior art disclosures only rely on pH gradients to accumulate the more soluble salt forms within the aqueous spaces which already contain acid or base.

Dissociation

When the pH of the lipid dispersion with the compound substantially dissolved (as the more soluble salt form or as ion-pairs) in the external aqueous medium is changed or restored to between pH 6 and pH 8, between pH 6.5 to 7.5, or between pH 7.2 to 7.4, the resulting dissociated (lipophilic) form may precipitate and spontaneously transfer to lipid particles thereby allowing further drug dissociation to establish equilibrium. The pH for association (solubilizing) in lipid particles depends on the pKa and Log P of the compound. Provided that there is substantial amount of the compound associated with the lipid when the system is at equilibrium, the final pH is between pH 6 and 8, or pH 7.2 to 7.4. Substantial refers to at least 30%, or at least 80% by weight. To facilitate transfer to the lipid particles, the temperature may be raised above ambient conditions where appropriate.

Referring to the Normogram in “Liposomes, A Practical Approach” (R.R.C. New, IRL Press at Oxford University Press), small unilamellar liposomes (SUVs) with 30 nm sdiameter and comprising 100 mg/ml lipid have the capacity to trap 60 μl in the internal aqueous space.

The lipophilic form of phenytoin is a weakly acidic, poorly soluble compound at pH 7.0 and the solubility in water is 37 μg/ml (Martindale The Extra Pharmacopoeia). In view of the absence of precipitates in the loaded liposome suspension at pH 7.0 in Example 4 below, the drug can only be held in lipid bilayers, internal aqueous compartments and/or external medium. It is found that the solution of phenytoin Na in 1M NaOH above pH 10 precipitates at pH 7.0, as shown in the blank control without liposomes. It is reasonable to assume that at pH 7.0 given that the liposome dispersion containing the drug does not cause precipitate formation, the phenytoin is most likely essentially carried in the lipid domain of the liposomal membrane. On the other hand it may be argued that the 2.5 mg drug internalized in Example 4 is somehow contained in the internal aqueous compartments (60 ul) then the solubility of phenytoin would be equivalent to 416 mg/ml. This amount if carried in the liposome aqueous phase is much higher than the solubility of phenytoin i.e. 37 μg at pH 7.0. Clearly, because of absence of precipitates in the loaded liposome suspension at pH 7.0, the lipophilic free acid form of phenytoin is more likely to be carried in the lipid domains rather than in the aqueous compartments.

Using dear liposome dispersions, the progress i.e., absence of precipitated drug particles may be monitored by visual assessment and confirmed by comparing the optical light transmission before and after pH changes and adjustments, and/or by comparing the precipitation behavior of the compound without or in the presence of the lipid particles.

Thus visual assessment is a useful guide to monitor transfer of the lipophilic compound from aqueous to liposomes under the conditions and pH employed. Visual assessment may be supported by comparing the transmission optical density (OD) measurements before and after the compound is added when the system is at equilibrium. Transfer to liposome particles is at equilibrium when the light transmission of the loaded dispersion at a wave length in the range of 600-800 nm does not change using ⅔ consecutive readings. The figures should be at least 80% of the initial transmission value of the blank dispersion which is used as a control. It reflects that at least 30% by weight of the compound is associated with the lipid particles. When the liposome dispersions are not sufficiently transparent the loading process can nevertheless be followed by e.g. using microscopy techniques to detect unassociated drug particles.

When the lipid dispersion is at a pH 6 to 8, the lipophilic form which is partitioned in the lipid may remain on the extensive surfaces of the lipid particles through charge and physical forces. The compound may be associated in the lipid domains of liposomal membranes, oil droplets, mixed micelles, or micelles. It is likely that because of much higher lipid affinity and solubility (log P) of the lipophilic form in lipid domains, the compound is “protected” and less subject to pH variations. Thus, the dosage form may have a useful shelf life of at least 4 weeks, or more after preparation without precipitation of large insoluble particles. The method is suited for extemporaneous preparation such as in a kit for use in situ as required by mixing the contents of the containers.

Extemporaneous embodiments may comprise two-container and three or more container applications in a kit.

Two container embodiments comprise a first container with the lipid dispersion and a second container with the compound dissolved in alkaline or acid medium.

Three vial embodiments may comprise a first container with the lipid dispersion, a second container with the powdered compound and a third vial with acid, alkaline or buffered medium.

The lipid dispersion may be neutral or buffered.

The contents of the containers may be added or mixed according to the method described wherein the pH change for forming the more soluble salt form is pH <6 or >8, optionally carried out in situ in the presence of lipid particles. For spontaneous lipid association the pH of the dispersion is adjusted to between pH 6 and pH 8 in the same or in a separate container.

Advantageously, the pH of the lipid dispersion is adjusted to the desired pH value before or after adding the poorly water soluble compound. The amount of compound solubilized in lipid particles and therefore free to interact with corresponding receptors may increase by a factor of 2, usually by at least 5, or by a factor greater than 10, simply by manipulating the pH of the aqueous medium to increase lipid solubility. Since the compound is solubilized or colloidally associated in lipid particles, bioavailability may be improved and higher concentrations may be delivered to target organs.

Solid dosage forms a composition which may be used for improving the solubility of pH sensitive poorly water soluble compound in a solid carrier dispersed in phospholipid is prepared by removing water from the aqueous medium prior to the final adjustment of pH between pH 6 and pH 8 thereby defining a solid composition. The solid composition comprising the compound uniformly dispersed in lipid particles or contiguous lipid matrix may be processed as e.g. capsules, compacts or pellets suitable for oral use. The compositions hydrate or dissolve in the stomach or they may be enteric coated for protection.

Additionally the solid composition may contain acid or alkaline powder components. The solid compositions are subject to pH changes in the GI track, enabling spontaneous solution of the compound in the lipid in intestinal or simulated intestinal fluid or a biorelevant medium that is between pH 6 and pH 8 thereby solubilizing at least 30% by weight of the poorly water soluble compound in lipid particles in the medium.

Alternatively, a composition suitable for oral use comprising the compound suspended in an aqueous dispersion or emulsion of phospholipids at pH 6-8 may be administered.

Confirmation that the compound can be solubilized in intestinal fluid in vivo is obtained by using a surrogate dissolution medium such as simulated intestinal fluid or biorelevant dissolution media which have been validated for in vivo/in vitro correlation,

pH Sensitive Compounds

The target ratio of drug to lipid is typically within the range 1:2 and 1:200, 1:1 to 1:100, or 1:5 to 1:50 parts by weight. Higher amounts of lipid may increase drug load when the system is at equilibrium. The composition may include from 1% to 5% by weight of surfactants, hydrophilic liquids or solvents to facilitate association with the lipid particles.

Examples of poorly soluble compounds include lipophilic compounds which are acidic or basic compounds. They contain polar groups which can be deprotonated or protonated e.g. carboxylic or sulphonic acid or phenolic groups and primary, secondary or tertiary amino groups. The compounds may be amino acids or amino acid based such as peptides, polypeptide and proteins, wherein the amino acids are exclusively or predominantly hydrophobic illustrated by leucine and valine. The acidic compound may be in the form of an alkali metal salt such as sodium salt and the like, and the basic compound may be in the form of an inorganic acid salt such as hydrochloride, borate, and the like and organic acid salt such as acetate, citrate, mesylate, malonic, propionate, ascorbate and the like.

The compounds may be employed in but not limited to diagnostic, therapeutic, or other applications covering e.g. CNS, oncology, cardiovascular diseases, metabolic, dermatology and antibiotic therapy, infectious diseases which includes human immuno deficiency infection (HIV) and respiratory syncytial virus infection (RSV).

The compound may be added as an aqueous dispersion or a solution in acid, alkaline or buffer to the lipid dispersion. The aqueous solution may contain a pharmaceutically acceptable water miscible solvent or liquid e.g. ethanol, PEG 100, 200, 300, 400, propylene glycol, NMP (N-methylpyrrolidone), DMA (N, N′-dimethylacetamide), DMSO (dimethylsulphoxide), DMI (dimethylimidazole) or mixtures thereof.

The invention is suitable for solubilizing compounds which are poorly soluble in water at essentially neutral pH between pH 6 and 8. The compounds may be weakly acid, basic or amphoteric with at least one ionizable or pH sensitive group. Suitable compounds may have pKa values between 4 and 10, between 5 and 9 or between 6 and 8.

Particular poorly water soluble compounds are characterized by:

a) low water solubility at pH 6-8,

b) high Log P between pH 6 and pH 8

c) high lipid solubility allowing substantial association with lipid carriers

d) at least one pH dependent ionizable group.

Lipid Dispersions

The lipid particles in the dispersion may consist of liposomes, micro emulsions, micelles, mixed micelles and mixtures thereof. The dispersions may be vesicular or non vesicular and include oil-in-water type emulsions or micro and macro emulsions, unilamellar or multilamellar liposomes, cubosomes, micelles, mixed micelles, bilayered and monolayered micro particles. The dispersion is selected from the group of optically clear lipid dispersions consisting of single unilamellar vesicles (SUV), cubosomes, oil-in-water type micro emulsions, micelles, mixed micelles, bilayered and monolayered micro particles and mixtures thereof. The dispersions are optically clear for parenteral administration.

The lipid dispersion comprises between 0.5% w/w to 25% w/w, below 20% w/w, or between 5% w/w to 15% w/w of at least one membrane lipid such as a phospholipid in the lipid mixture. Higher drug concentration may be obtained by increasing the amount of lipid available for drug association when the system is at equilibrium i.e. increasing the amount of lipid dispersed in the aqueous medium.

Preparation of Optically Clear Lipid Dispersion

A typical embodiment involves preparing the aqueous dispersion containing preformed empty or placebo lipid particles in bulk or large volumes in the first instance for transfer into smaller containers, ready to use. The aqueous medium may contain buffers, such as phosphate, citrate/phosphate, TRIS or histidine buffers.

A method that gives uniform lipid particles and optically clear lipid dispersion may be employed such as sonication and using high mechanical impact dispersators, colloid mills. High pressure homogenization and extrusion methods may be used. Some mixed micelles and micelles consisting of e.g. bile acid salts are normally naturally clear dispersions and may not require similar high pressure homogenizing. The lipid dispersion may be sterilized by aseptic filtration or end sterilization by autoclaving the loaded lipid dispersion in the final container.

It should be understood that the lipid dispersion is characterized in terms of particle size, lamellarity, optical clarity, type of phospholipids and/or surfactants. For screening studies involving exposure in organisms, the lipid type and the charge carried; presence of ligands such as pegylation; structure and size of the lipid particles such as lamellarity; can result in different organ distributions and concentrations. Where the pharmacokinetics of a new compound is not yet fully elucidated, standardized lipid particles with known organ distribution and clearance are desired in ADME studies to rule out or control potential interference and/or masking due to lipid carriers.

Lipid

The lipid used for preparing dispersions may comprise a diacyl lipid or a monoacyl lipid on its own or it may contain mixtures of the monoacyl and diacyl components obtained by controlled enzyme hydrolysis.

The lipid comprises at least one lipid with amphipathic properties from the class of membrane lipids comprising phosphoglycerides or sphingolipids or a combination of said lipids. Alternatively, in place of or in partial replacement of amphipathic membrane lipids, particularly when oral compositions are envisioned, the lipid composition may contain synthetic surfactants such as polysorbate 80 and non ionic polyglycerol esters and ethers (Solutol HS). The lipid dispersion contains at least one membrane lipid such as at least one phospholipid of the formula

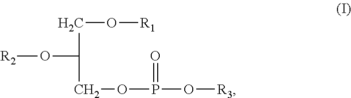

wherein

R₁ represents C₁₀-C₂₀acyl;

R₂ represents hydrogen or C₁₀-C₂₀acyl;

R3 represents hydrogen, 2-trimethylamino-1-ethyl, 2-amino-1-ethyl, Ci-C₄alkyl, C₁-C₅alkyl substituted by carboxy, C₂-C₅alkyl substituted by carboxy and hydroxy, C₂-C₅alkyl substituted by carboxy and amino, an inositol group or a glyceryl group or a salt of such compound.

The phospholipid may be neutral or it may be charged. It may be a double fatty acid chain or a single fatty add chain amphipath.

Examples of neutral phospholipids with double chains are phosphatidylcholine (PC), phosphatidylethanolamine (PE) and sphingomyelin.

Examples of charged phospholipids are phosphatidic add (PA), phosphatitdyl inositol (Pi) and phosphatidylserine (PS) and phosphatidylglycerol (PG). The hydrocarbon chain can either be unsaturated or saturated and can have between 10 and 24, or 14 and 18 carbon atoms.

The single chain lipid is the monoacyl derivative of a neutral or charged phospholipid, but it can also be the monoacyl derivative(s) of glycolipids and sphingolipids.

Deacylation may be carried out by phospholipase A1, phospholipase A2, or by chemical means. Deacylation by phospholipase A2 enzyme hydrolysis may also be used. The hydrocarbon chain can either be unsaturated or saturated and can have between 10 and 24, or 14 to 18 carbon atoms.

The lipids may be derived from natural plant, or animal or microbiological sources, synthesized or partially synthesized, including polyethyleneglycol (PEG) derived monoacyl phospholipids, eg. pegalated monoacyl phosphatidyl ethanolamine.

Bilayer lipids, such as glycolipids, ceramides, gangliosides and cerebrosides can be used in place of, or in partial replacement of phospholipids. A bilayer lipid is phosphatidylcholine (PC). Preferred diacyl phosphatidylcholine is soy PC, followed by Egg PC, POPC, and OOPC. Monacyl counterpart is enzyme modified (Phospholipase A2) soy PC, followed by Egg PC, 1-palmitoyl PC, 1 oleoyl PC, 1-stearoyl PC. Regardless of the type of lipid or lipid mixture desired, essentially the specifications are defined within set limits and ranges, with the key components specified qualitatively and quantitatively for standardization and control reproducibility of the lipid dispersions described in this invention.

Bile Salts:

The lipid composition may further comprise phospholipids and/or a bile salts acid or salt such as sodium tauroglycholate and sodium glycocholate and mixtures thereof. Similarly the specifications are controlled to ensure standardization of the lipid dispersion comprising micelles or mixed micelles.

Acids, Alkalis and Buffers for pH Manipulation

pH changes to the desired value to increase water solubility for loading or to adjust the pH of the lipid dispersion for lipid association may be by addition of 0.1 N HCl or NaOH or a buffer solution. Suitable buffers are for example phosphate buffer, Phosphate/citrate buffer, Tris or Histidine solutions.

Other Pharmaceutically Acceptable Excipients

Excipients for an oil-in-water emulsion are acylglycerols, such as, e.g., short chain and medium chain triglycerides, soy bean oil and nonpolar alcohols with high molecular weight. Pharmaceutically acceptable excipients may be present, either as stabilizers or preservatives, particularly in solid oral dosage formulations with or without enteric coating or protection. Furthermore, enteric protection may be included along with stabilizers in the lipid dispersions. Examples of stabilizers that may be included in the lipid dispersions are isotonic and buffering agents, e.g. sugars and salts, or anti-oxidants, e.g alpha tocopherol acetate, ascorbyl palmitate, BHT and BHA. Examples of preservatives are anti-microbials e.g, methyl paraben and butyl paraben. Cryoprotectants, binders, desintegrants, fillers are e.g. starch, polyvinyl pyrrolidones, carboxymethylcellulose, poloxamers. Examples of hydrophilic liquids are polyethylene glycol 3000 and polyethylene glycol 4000. Examples of osmotic regulators are glycerol, sodium chloride, potassium chloride, sugars such as glucose, mannitol, lactose, trehalose and saccharose. Examples of enteric coatings and protectants are based on anionic polymers of methacrylic acid and methacrylates which dissolve between pH 5.5 to 7.

The invention describes a method of preparing lipid compositions by associating pH sensitive compounds which maybe weak acids, bases or amphoteric compounds with at least one ionizable or pH sensitive group and low water solubility into preformed and standardized lipid particles by pH manipulation. Drug association is carried out in situ by changing the pH of the aqueous medium to pH 6-8 wherein the compound spontaneously partitions into the lipid particles. Drug association may also be carried out by exploiting pH changes in the GI tract. The lipophilic compound is solubilized by the lipid particles and may be used as such for in vitro and pharmacokinetic assessments or processed for oral, parenteral or topical therapy.

### EXAMPLES

### Example 1

A first aqueous suspension comprising SUVs is prepared in a micro-fiuidizer. The dispersion contains 100 mg phospholipid/ml in isotonic glycerol solution. It is optically clear with transmission of &gt;80%. PCS measurement indicates a mean particle size of ca 30 nm.

A second aqueous solution contains 100 mg diazepam dissolved in 1 ml of 1 M HCl at about pH 1. Diazepam has a pKa of 3.4, Log P oil/water 2.9; solubility 56 μg/ml in water at pH 7 and solubility 2148 μg/ml at pH 1.2.

50 μl of the second solution containing 5 mg diazepam/ml is added to 1 ml of the dear lipid dispersion.

Alternatively, rather than separately preparing a second aqueous solution containing diazepam, the drug is added to the first aqueous suspension containing liposomes and solubilized by adding 1M HCL at about pH 1.0.

The resulting dear acidic liposomal dispersion in either case with the drug in solution in the external phase is neutralized to pH 7.0 using NaOH. The liposomes remain dear without diazepam precipitates.

In a separate control and omitting phospholipid, 50 μl of a 100 mg diazepam/ml solution containing 5 mg diazepam is added to 1.0 ml isotonic glycerol solution. Neutralizing to pH 7.0 with NaOH precipitates out the diazepam as a cloudy suspension.

The results of the control experiment clearly show that the diazepam is solubilized in the liposomes at pH 7.0.

### Example 2

100 mg dipyridamole is dissolved in 1 ml of 1 M HCl. The compound has the following properties: pKa 6.4, Log P oil/water 1.5; solubility in water at pH 7: 5 μg/ml, solubility at pH 1.2: 1753 μg/ml at. 25 μl of the drug solution is added to 1.0 ml of a clear liposomal dispersion with particle size ca 30 nm comprising 100 mg phospholipid/ml in isotonic glycerol solution prepared as in Example 1. Alternatively the drug may be suspended directly in the liposome suspension and solubilized with acid around pH 1.2 as in Example 1.

The acidic clear liposomal dispersion containing 2.5 mg dipyridamole/ml is neutralized to pH 7.0 with NaOH and the liposomes remain clear. No precipitated dipyridamole is noticed.

in a control experiment 25 μl of the 100 mg dipyridamole/ml solution is added to 1.0 ml of isotonic glycerol solution to make up 2.5 mg dipyridamole/ml. After neutralization to pH 7.0 with NaOH, the dipyridamole precipitates. The results of the control experiment show that the dipyridamole is solubilized in the neutralized liposomes at pH 7.

### Example 3

100 mg ketoconazole is dissolved in 1 ml of 1 M HCl. Ketoconazole possesses 2 pKa values: pKa1: 2.9; .pKa2: 6.5; Log P oil/water: 4; solubility: 2 μg/ml in water at pH 7.0:, solubility; 2729 μg/ml at pH 1.2. As in Example 1, 50 μl of this solution is added to 1.0 ml of a previously prepared, dear liposomal dispersion comprising 100 mg phospholipid/ml in isotonic glycerol solution in which the liposomes have a particle size of ca 30 nm. Alternatively the drug may be suspended directly in the liposome dispersion and solubilized with acid at pH about 1.2 as in Example 1.

The acidic clear liposomal dispersion containing 5 mg ketoconazole/ml is neutralized to about pH 7 with NaOH and the liposomes stay clear and no precipitated ketoconazole can be seen.

Ina control experiment 50 μl of the 100 mg ketoconazole/ml solution is added to 1.0 m of isotonic glycerol solution to make 5 mg ketoconazole/ml. After neutralization to pH 7.0 with NaOH, the poorly soluble ketoconazole precipitates. The control experiment provides support that the drug is solubilized in the neutralized liposomes.

### Example 4

Phenytoin with the following properties pKa 8.3, Log P oil/water 2.3; solubility in water at pH 7.0: 37 μg/ml, solubility at pH 10-pH13: Phenytoin sodium is freely soluble according to Martindale (between 1:1 and 1:10).

100 mg of drug is dissolved in 1 M NaOH at above pH 10.0.

25 μl of this solution is added to 1.0 ml of a clear liposomal dispersion containing 100 mg phospholipid in isotonic glycerol solution .The liposomes have a mean particle size of ca 30 nm.

The clear basic liposomal dispersion containing 2.5 mg phenytoin/ml is neutralized to circa pH 7.0 with HCl and the liposomes stay clear and no precipitated phenytoin can be observed.

In a control experiment 25 μl of the 100 mg phenytoin/ml solution in 1 M NaOH is added to 1.0 ml of isotonic glycerol solution to make up 2500 μg phenytoin/ml. After neutralization to around pH 7.0 with HCl, the phenytoin precipitates. The results of the control experiment without lipid particles prove that 2500 μg phenytoin is solubilized in the liposomes at around pH 7.0 although the aqueous solubility at pH 7.0 is 3.7 μg/ml.

1-15. (canceled)

16. A method of solubilizing a biologically active, poorly water soluble compound with one or more ionizable or pH sensitive groups and with low water solubility between about pH 6 and pH 8, comprising:
providing an aqueous medium in the form of a lipid dispersion;
adding a biologically active, poorly water soluble compound to the aqueous medium;
adjusting a pH of the aqueous medium so that a higher amount of the poorly water soluble compound is being dissolved than at essentially neutral conditions;
mixing the aqueous medium to produce a mixed medium; and
adjusting a pH of the mixed medium to between about pH 6 and pH 8 to transfer the poorly water soluble compound to lipid particles in the mixed medium to form a lipid dispersion containing the poorly water soluble compound.

17. The method of claim 16, wherein the providing the aqueous medium comprises forming the lipid dispersion in the aqueous solution.

18. The method of claim 16, further comprising changing the pH of the aqueous medium for a basic compound to below about pH 6 or for acidic compound to above about pH 8.

19. The method of claim 16, wherein the transfer of the he poorly water soluble compound to lipid particles occurs at a pH between an initial pH and a subsequently adjusted pH.

20. The method of claim 16, further comprising adjusting the pH of the mixed medium to a final pH of between about 7.2 and 7.4 for use as a parenteral or intravenous dosage form.

21. The method of claim 16, further comprising adding the poorly water soluble compound in a powder form to said aqueous medium containing the lipid dispersion at about pH 7 before changing the pH to below about pH 6 or above about pH 8.

22. The method of claim 17, further comprising providing the aqueous medium at a desired pH below about pH 6 or above about pH 8 before adding the poorly water soluble compound to obtain a more soluble form in situ.

23. The method of claim 22, further comprising providing the aqueous medium with a buffer to form a buffered lipid dispersion that has sufficient buffering capacity so that after adding the poorly water soluble compound, that is dissolved or partially dissolved in an acid or alkaline medium or in a more soluble salt form, the pH of a resulting composition is between about pH 6 and pH 8.

24. The method of claim 23, further comprising dissolving the poorly water soluble compound in one of an acid pH solution below pH 4 or an alkali pH solutions above pH 8 before adding to the buffered lipid dispersion, and adjusting the pH of the buffered lipid dispersion by at least one pH unit above a pH of the alkali pH solution or below a pH of the of the acid pH solution that contains the dissolved or partially dissolved poorly water soluble compound.

25. The method of claim 16, further comprising simultaneously mixing the aqueous medium to produce a mixed medium and adjusting the pH of the mixed medium to between about pH 6 and pH 8 to transfer the poorly water soluble compound to lipid particles in the mixed medium to form a lipid dispersion loaded with the poorly water soluble compound.

26. The method of claim 25, further comprising removing water from the mixed medium to prepare a solid composition.

27. The method of claim 16, further comprising performing at least one pH change followed by a subsequent removal of the aqueous medium, and performing a final pH change of between about pH 6 and pH 8 in an intestinal fluid or biorelevant media.

28. The method of claim 16, further comprising removing the aqueous medium after the first pH change to obtain a solid composition, and further comprising dissolving the solid composition in another aqueous medium and changing pH wherein a final pH change is carried out between about pH 6 and pH 8 in an intestinal fluid or biorelevant media,

29. The method of claim 16, further comprising dispersing or partially dispersing the poorly water soluble compound in a solid carrier together with at least one lipid or lipid particles by removing water after the first pH change to produce a solid composition, and further comprising dissolving said composition in another aqueous medium, followed by further pH changes wherein a final pH change is carried out between about pH 6 and pH 8 in an intestinal fluid or biorelevant media.

30. The method of claim 16, further comprising providing a kit having a first container containing the lipid dispersion in the aqueous medium, a second container containing a second aqueous medium in the form of an alkaline, buffered or acid medium, and the poorly water soluble compound dissolved in the second aqueous medium or contained in a third container in powder form.

31. The method of claim 30, further comprising using the kit to solubilize a biologically active poorly water soluble compound with one or more ionizable or pH sensitive groups and low water solubility between about pH 6 and pH 8.
